(19) United States
(12) Patent Application Publication
(10) Pub. No.: US 2006/0078608 A1
(43) Pub. Date: Apr. 13, 2006

(54) Flavored gelatin capsule and method for producing said capsule

(76) Inventors: Jonathan Gilinski, Aventura, FL (US); Amira Isolina Ladron de Guevara Sanchez, Barranquilla (CO)

Correspondence Address: PROSKAUER ROSE LLP;PATENT DEPARTMENT, 1585 BROADWAY, NEW YORK, NY, 10036-8299, US

(21) Appl. No.: 11/243,582

(22) Filed: Oct. 5, 2005

Related U.S. Application Data
(60) Provisional application No. 60/617,524, filed on Oct. 8, 2004.

(51) Int. Cl. A61K 9/64

(52) U.S. Cl. 424/456

(57) ABSTRACT

The present invention is directed to a flavored capsule suitable for encapsulating a dose and a method for making a flavored capsule. In one embodiment, a flavored capsule comprises a gelatin, water and a flavoring. The flavoring may be present in the flavored capsule in a concentration of between about 1% and about 4% by weight.

### RELATED APPLICATION

This application claims the benefit of U.S. Provisional Application No. 60/617,524, filed Oct. 8, 2004, the entire disclosure thereof is incorporated by reference herein.

### BACKGROUND OF INVENTION

The taste of many medicinal and nutritive components can be quite distinctive and potentially unpleasant. Improvements in the taste of certain drugs and nutritional supplements can lead to higher compliance by consumers. A higher compliance will result in greater commercial success for the drug and supplement manufacture, and in increased health and well being particularly to consumers.

Attempts to create a flavored capsule have been limited by the requirement to use high amounts of softeners, including hydrogenated saccharide, synonymous with polyol (polyalcohol) or gelatin softeners, and consequently lower amounts of flavoring. U.S. Pat. No. 6,652,879, to Opheim, teaches a flavored gelatin capsule in which the concentration of flavoring is in the range between about 0.25% and about 1.5% of the gelatin capsule, and the capsule contains a gelatin softener in a concentration of 10% to 35% of the capsule. U.S. Pat. No. 6,641,837, also to Opheim, teaches a flavored vegetable starch capsule in which the concentration of flavoring is in the range between about 0.25% and about 1.5% of said flavored vegetable starch capsule, and wherein one polyol, such as glycerol or sorbitol, is present in the range of between 10% and 35% of said flavored vegetable starch capsule. U.S. Pat. No. 5,264,223, to Yamamoto, describes the inclusion of a flavor in a capsule, but does not specify the concentration that can be provided by its teachings.

Providing a capsule with a greater amount of flavoring and a method of making a capsule with a greater amount of flavoring will be beneficial to improving consumer consumption of medicinal and nutritional supplements resulting in better health and overall well-being.

### SUMMARY OF THE INVENTION

The present invention is directed to capsule formulations and methods of manufacturing capsules. In one embodiment, a flavored gelatin capsule suitable for encapsulating a dose is provided. This flavored gelatin capsule includes a greater amount of flavoring than the capsules described above without including softener. In another embodiment, a method for making a flavored gelatin capsule is provided.

### DETAILED DESCRIPTION OF THE PREFERRED EMBODIMENTS

A first embodiment of the present invention provides a flavored gelatin capsule. In alternative embodiments, other types of capsules that benefit from flavoring may be used instead of the gelatin flavored capsules described herein.

In one embodiment of the present invention, a flavored gelatin capsule is provided that is suitable for encapsulating a dose. The capsule includes about 75% to about 85% by weight of a gelatin, about 13% to about 16% by weight water and about 1% to about 4% by weight of a flavoring. In a preferred embodiment, the capsule includes about 80% by weight of a gelatin, about 15% by weight water and about 1-4% by weight of a flavoring.

The capsule may be used to encapsulate a dose, such as, but not limited to, nutritional supplements, vitamins, minerals, over-the-counter drugs, and prescription drugs. Any nutritional supplement may be so encapsulated, including, but not limited to, an amino acid, vitamin, or animal product, such as acetyl-1-carnosine, alpha lipoic acid, amylase, androstendiol, androstendione, arginine, ascorbic acid, B vitamin, beta-carotene, biotin, bromelain, calcium, chicken collagen, chitosan, choline, chondroitin, coenzyme Q10, creatine, dehydroepiandrosterone, diethylmethylaminoethanol, dihydroepiandsterone, dimethylglycine, DMSO, gammahydroxybutric acid (GABA), glucosamine, glutamine, glutathione, hyaluronic acid, hydroxytryptophan, indium, isoleucine, 1-carnitine, lactoferrin, lecithin, leucine, lipase, lumbrokinase, lutein, magnesium, melatonin, Methylcobalamin, methylsulfonylmethane, MGN 3, ornithine, pancreatin, panthethoic acid, papain, para-amino benzoic acid (PABA), phenylalanine, phosphatidylcholine, phosphatidylserine, potassium, pregnenalone, protease, retinoic acid, retinol, s-adenosyl-methionine, selenium, taurine, theanine, thymase, tocopherol, trimethylglycine, tryptophan, tyrosine, valine, vinpocetine, vitamin D, vitamin A, zeathanthine, zinc, and their nutraceutically acceptable salts, ethers, esters, acid, or other derivatives.

A nutritional supplement may also be chosen from herbal preparations, such as the leaf, root, or extract of a plant, including, but not limited to, artichoke, bilberry, bioflavonoid, boswella, bupleurium, chamomile, chlorophyll, cranberry, damiana, echinacea, essiac, garcinia cambogia, garlic, germanium, ginger, gingko, ginseng, goldenseal, grape seed, green tea, hawthorne berry, hesperidin, hops, horse chestnut hydrangea, hypericum, indole-3-carbinol, licorice, lycopene, nettle root, peppermint, periwinkle, policosanol, psyllium, pygeum, quercetin, raspberry, resveratol, rutin, sassafras, saw palmetto, silymarin, tribulus terestris, turmeric, valerian, wild yam, and their nutraceutically acceptable salts, ethers, esters, acid, or other derivatives.

The gelatin may be any suitable type, kind and/or grade of gelatin. In one embodiment, type B pharmaceutical gelatin is utilized. This type B gelatin is obtained by the partial hydrolysis of collagen derived from the connective tissue of the bovine livestock. It is a translucent, amorphous pellet that is clear yellow color to dark yellow color, soluble in hot water and has a characteristic odor. In aqueous solution, the gelatin jellifies between 25° C. and 26° C. at 10% of concentration. Its pH is about 5.0 to about 6.4, bloom value is about 260 g to about 300 g and it operates at a temperature of about 115° F. to about 130° F. In one embodiment of the present invention, a gelatine solution is utilized. However, it is also suitable to use gelatin in the form of a sheet, granule or powder.

The flavoring includes a flavor that may be one of, or a combination of, suitable flavors known in the art, including, but not limited to, berry, strawberry, grape, mint, orange, blueberry, chocolate, cocoa, lemon, nut, almond, cashew, macadamia nut, coconut, coffee, butter, peanut, meat, bacon, chicken, beef, liver, blackberry, raspberry, peach, lime, banana, chili pepper, spicy, pepper, peppermint, cinnamon, vanilla, eucalyptus and pineapple. Preferably, the flavor is mint, blackberry, raspberry, spicy, cinnamon, chicken, meat, coffee, lime, bacon, orange, or grape. It is suitable to use either liquid or powder flavoring.

The capsule may optionally include one or more lubricants, such as, but not limited to, silicon dioxide, titanium dioxide, calcium phosphate, calcium sulfate, sodium laurel sulfate, silicified microcrystalline cellulose, magnesium stearate, and talc. Various silicones are available from manufacturers known to those skilled in the art, such as United Chemical Technologies, Inc., of Bristol, Pa. and other suitable lubricants are available from numerous manufacturers including Bioclean Impex of India. In one embodiment of the present invention, the capsule includes up to about 0.05% by weight of sodium laurel sulfate.

The capsule may optionally include one or more colorants or dyes. For example, a dye solution may be prepared by weighing dye powders and dissolving them in hot water. In one embodiment of the present invention, the flavored gelatin capsule includes up to about 4% by weight of a colorant.

Reference is now made to methods that may be used for making a flavored gelatin capsule. In one embodiment of the present invention, the manufacturing steps include combining a gelatin and water to form a solution, melting and de-bubbling the gelatin solution, combining a flavoring with the gelatin solution, transferring the solution to a capsule-manufacturing machine, dipping mold pins into the solution to form a film around the pins, drying the film, stripping the capsule parts of cap and body from the pins and joining the cap and body together. A dye solution may be added to the gelatin solution after the gelatin solution is melted and de-bubbled.

In another embodiment of the present invention, a solution of gelatine is prepared by weighing gelatine and adding it to water. The gelatine solution's concentration level is about 32.67%. The gelatine solution is added to stainless steel jacketed melt tanks and is melted at a temperature of about 115 degrees Fahrenheit to about 130 degrees Fahrenheit. A dye solution is prepared by weighing dye powders and dissolving them in hot water. The dye solution is added to the gelatin solution in the stainless steel tanks after the gelatine solution has been melted and de-bubbled. A flavoring solution is prepared with food-grade flavor powder and water, or alternatively a flavored liquid base is weighed, about 4% to about 15% by weight. The flavoring solution or liquid base is added to the gelatine solution after the gelatine solution has been melted and de-bubbled. Additional water is added after the gelatine solution is melted and de-bubbled to achieve a specific viscosity. The viscosity achieved varies from about 450 to about 800 cps and depends on the intended capsule size. The solution is transported to a machine that produces gelatine capsules. Molds, or pins, are dipped into the solution to form the capsule parts of cap and body. After the solution is adhered to the molds, it is submitted to a drying process with heat and dry air. The formed parts (cap and body) are stripped from the molds using a lubricant. Thereafter, the cap and body are joined together.

Lubricants including, but not limited to, sodium lauryl sulfate, silicon dioxide and titanium dioxide, may be weighed and added to the gelatin solution after melting and prior to de-bubbling. To ease the stripping of the formed parts (cap and body) from the molds, lubricants may also be added directly on the pin molds before the molds are dipped into the gelatin solution.

Additional water, one or more lubricants and one or more dye solutions may be added after melting and de-bubbling to achieve the color and viscosity needed for the type and size of capsules desired. The viscosity may vary from about 450 to about 800 cps.

The level of concentration of the amount of flavor added to the gelatine solution depends on the type of flavor desired. This concentration level may vary from about 4% to about 15% depending on the flavor desired.

While this invention has been particularly shown and described with references to preferred embodiments thereof, it will be understood by those skilled in the art that various changes in form and details may be made therein without departing from the spirit and scope of the invention as defined by the appended claims. Those skilled in the art will recognize or be able to ascertain using no more than routine experimentation, many equivalents to the embodiments of the invention described herein. Such equivalents are intended to be encompassed in the scope of the claims.

What is claimed is:

1. A flavored gelatin capsule suitable for encapsulating a dose, said capsule comprising:
a. about 75% to about 85% by weight of a gelatin;
b. about 1% to about 4% by weight of a flavoring; and
c. about 13% to about 16% by weight water.

2. The flavored gelatin capsule of claim 1, wherein the capsule comprises about 80% by weight gelatin, about 1% to about 4% by weight of flavoring and about 15% by weight water.

3. The flavored gelatin capsule of claim 1, further comprising an encapsulated dose.

4. The flavored gelatin capsule of claim 3, wherein the encapsulated dose is selected from the group consisting of nutritional supplements, over-the-counter drugs and prescription drugs.

5. The flavored gelatin capsule of claim 1, wherein the flavoring comprises a flavor selected from the group consisting of berry, strawberry, grape, mint, orange, blueberry, chocolate, cocoa, lemon, nut, almond, cashew, macadamia nut, coconut, coffee, butter, peanut, meat, bacon, chicken, beef, liver, blackberry, raspberry, peach, lime, banana, chili pepper, spicy, pepper, peppermint, cinnamon, vanilla, eucalyptus and pineapple.

6. The flavored gelatin capsule of claim 1, wherein the flavoring comprises a flavor selected from the group consisting of mint, blackberry, raspberry, spicy, cinnamon, chicken, meat, coffee, lime, bacon, orange, or grape.

7. The flavored gelatin capsule of claim 1, further comprising a lubricant.

8. The flavored gelatin capsule of claim 7, wherein the lubricant is sodium laurel sulfate.

9. The flavored gelatin capsule of claim 1, further comprising one or more colorants.

10. A flavored gelatin capsule suitable for encapsulating a dose, said capsule comprising:
a. a gelatin;
b. a flavoring; and
c. water;
wherein the flavoring is present in said gelatin capsule in a concentration of between about 1% and about 4% by weight.

11. The flavored gelatin capsule of claim 10, wherein the capsule comprises about 75% to about 85% by weight gelatin and about 13% to about 16% by weight water.

12. The flavored gelatin capsule of claim 10, wherein the capsule comprises about 80% by weight gelatin and about 15% by weight water.

13. The flavored gelatin capsule of claim 10, wherein the flavoring comprises a flavor selected from the group consisting of mint, blackberry, raspberry, spicy, cinnamon, chicken, meat, coffee, lime, bacon, orange, or grape.

14. A method for manufacturing a flavored gelatin capsule comprising the steps of:
(a) combining a gelatine and water to form a solution;
(b) melting and de-bubbling the gelatine solution;
(c) combining a flavoring solution to the gelatine solution;
(d) dipping mold pins into the solution to form capsule parts of cap and body;
(e) drying the film that has formed around the pins;
(f) stripping the cap and body from the pins; and
(g) joining the cap and body together.

15. The method of claim 14, further comprising the step of combining a dye or dye solution with the gelatine solution after step (b) and prior to step (d).

16. The method of claim 14, wherein the flavoring solution comprises a flavoring present in a concentration of between about 4% and about 15% by weight.

17. The method of claim 14, wherein the flavoring solution comprises a flavor selected from the group consisting of berry, strawberry, grape, mint, orange, blueberry, chocolate, cocoa, lemon, nut, almond, cashew, macadamia nut, coconut, blueberry, blackberry, raspberry, peach, lime, banana, chili pepper, pepper, cinnamon, coffee, butter, peanut, peppermint, chicken, beef, liver, vanilla, eucalyptus and pineapple.

18. The method of claim 14, wherein the gelatine solution is prepared by weighing the gelatine and adding it to water so that the gelatine solution's concentration is about 32.67%.

19. The method of claim 14, wherein the flavoring solution is prepared by adding food-grade flavor powder to water so that the flavoring concentration in the solution is about 4% to about 15% by weight.

20. The method of claim 14, wherein the gelatine solution is melted at a temperature of about 115 degrees Fahrenheit to about 130 degrees Fahrenheit.
